(19) United States
(12) Patent Application Publication
(10) Pub. No.: US 2014/0134751 A1
(43) Pub. Date: May 15, 2014

(54) DIAGNOSTIC METHOD

(71) Applicants: Simon D. Taylor-Robinson, London (GB); Shahid Aziz Khan, Middlesex (GB); Mohamed I.F. Shariff, Middlesex (GB)

(72) Inventors: Simon D. Taylor-Robinson, London (GB); Shahid Aziz Khan, Middlesex (GB); Mohamed I.F. Shariff, Middlesex (GB)

(73) Assignee: IMPERIAL INNOVATIONS LTD.

(21) Appl. No.: 13/982,835

(22) PCT Filed: Jan. 31, 2012
(86) PCT No.: PCT/GB2012/000100

§ 371 Date: Oct. 9, 2013

(30) Foreign Application Priority Data
Jan. 31, 2011 (GB) 1101667.2

(51) Int. Cl. G01N 33/53; G01N 33/50

(52) U.S. Cl. CPC G01N 33/5308; G01N 33/50
USPC 436/501; 436/106; 436/112; 436/129

(57) ABSTRACT

Disclosed herein is a method for diagnosing hepatic cancer by analysing a sample and determining the level of at least one compound selected from the group consisting of glycine, trimethylamine-N-oxide, hippurate and citrate and comparing the levels in the sample with control levels. The analysis of the sample can involve the determination of a profile for the sample. The methods disclosed are useful in distinguishing between a patient having hepatic cancer and a patient having cirrhosis.

### TECHNICAL FIELD

This invention relates to diagnostic methods for identifying subjects suffering from hepatic cancer.

### BACKGROUND OF THE INVENTION

Hepatic cancer may take the form of primary hepatic cancer which is considered to be cancer which originates from the liver, or secondary hepatic cancer where the cancer originates in the liver and migrates to another organ. Hepatocellular carcinoma (HCC) is the most common form of primary hepatic cancer and is the third most common cause of cancer death worldwide^{1,2,3}. The disease is particularly prevalent in the developing world, and especially sub-Saharan Africa and Asia⁴, where several countries display a high incidence of over 20 cases for every 100,000 people. If the cancer cannot be completely removed, the disease is usually fatal within 3-6 months⁵. Symptoms of HCC can be very severe and include jaundice, bloating from ascites, easy bruising from blood clotting abnormalities, loss of appetite, unintentional weight loss, abdominal pain, especially in the upper-right part, nausea, emesis, and fatigue⁶.

Current methods of diagnosis include screening for HCC using serum α-fetoprotein (AFP), a fetal glycoprotein that is normally undetectable soon after birth. Most HCC secrete AFP, but AFP has poor sensitivity and specificity of less than 70%^{7,8,9,10}. Furthermore, AFP testing of serum can be prohibitively expensive and therefore unavailable in parts of Africa and Asia. Many other serum markers including des-gamma-carboxyprothrombin, anti-p53, gamma-glutamyl-transpeptidase and isoferritin are also used in screening for HCC, but like AFP display a low degree of sensitivity and specificity^{11,12}.

The problem of sensitivity and specificity of serum markers for HCC is further heightened by the fact that patients suffering with cirrhosis can be difficult to distinguish from patients suffering with HCC using current methods. Early diagnosis of HCC is very important, since lesions below 2 cm are curable with resection or transplant. There is therefore a real need in the art for the development of a test with a level of sensitivity and specificity high enough to distinguish between patients with HCC, cirrhosis and healthy controls, even at early stages of disease.

In addition to the problems involving low sensitivity and specificity of serum markers, the areas where the disease is most prevalent give rise to ethical considerations. In many African and Asian countries, religious beliefs prohibit the use of invasive techniques in screening for disease. It is also important that information from the test be available quickly after testing is carried out since patients in the developing world may live many days journey from the health clinic.

HCC can be diagnosed more accurately using CT scans, MRT scans and biopsy¹³. However, the costs and time involved with these techniques as well as the invasive nature of biopsy, mean that these tests are not suitable for developing areas where the disease displays the highest prevalence.

There is thus a need for the development of a method of diagnosing HCC which is both specific and sensitive, as well as being practical and available for use in both the developed and the developing world. A sensitive and specific test would allow for detection of the disease at an early stage, leading to a significant improvement in the prognosis for HCC sufferers.

### DISCLOSURE OF THE INVENTION

Accordingly, the invention provides a method for analysing a sample from a test subject comprising:

i) determining the level of at least one compound selected from the group consisting of glycine, trimethylamine-N-oxide, hippurate and citrate in the sample from the test subject; and
ii) comparing the level of the at least one compound determined in step i) to at least one control level, wherein the levels of the at least one compound are indicative of whether the subject has hepatic cancer.

Surprisingly, the inventors have found that the levels of glycine, trimethylamine-N-oxide, hippurate and citrate are significantly lower in samples taken from subjects with hepatic cancer compared with samples taken from healthy subjects. The levels of such compounds have never before been measured as part of a method for diagnosing hepatic cancer. The measurement of the levels of these particular compounds allows for sensitive and specific screening for hepatic cancer, and the fact that the compounds may be measured using a wide range of simple assays will aid diagnosis for hepatic cancer in the developing world where the disease is most prevalent.

The method of the invention may comprise i) determining the level of glycine in a sample from a test subject; and comparing the level of glycine determined in step i) to a control level.

The method of the invention may comprise i) determining the level of trimethylamine-N-oxide in a sample from a test subject; and comparing the level of trimethylamine-N-oxide determined in step i) to a control level.

The method of the invention may comprise i) determining the level of hippurate in a sample from a test subject; and comparing the level of hippurate determined in step i) to a control level.

The method of the invention may comprise i) determining the level of citrate in a sample from a test subject; and comparing the level of citrate determined in step i) to a control level.

The method of the invention may comprise determining the level of at least two compounds selected from the group consisting of glycine, trimethylamine-N-oxide, hippurate and citrate and comparing the level of the at least two compounds to a control level. In one embodiment, the method may comprise determining the level of glycine and trimethylamine-N-oxide and comparing the levels to control levels. In another embodiment the method may comprise determining the level of glycine and hippurate and comparing the levels to control levels. In another embodiment the method may comprise determining the level of glycine and citrate and comparing the levels to control levels. In another embodiment the method may comprise determining the level of trimethylamine-N-oxide and hippurate and comparing the levels to control levels. In another embodiment the method may comprise determining the level of trimethylamine-N-oxide and citrate and comparing the levels to control levels. In another embodiment the method may comprise determining the level of hippurate and citrate.

The method of the invention may comprise determining the level of at least three compounds selected from the group consisting of glycine, trimethylamine-N-oxide, hippurate and citrate and comparing the levels to control levels. In one embodiment, the method may comprise determining the level of glycine, trimethylamine-N-oxide and hippurate and comparing the levels to control levels. In one embodiment, the method may comprise determining the level of glycine, trimethylamine-N-oxide and citrate and comparing the levels to control levels. In one embodiment, the method may comprise determining the level of trimethylamine-N-oxide, hippurate and citrate and comparing the levels to control levels. In one embodiment, the method may comprise determining the level of glycine, hippurate and citrate and comparing the levels to control levels.

The method of the invention may comprise determining the level of glycine, trimethylamine-N-oxide, hippurate and citrate and comparing the levels to control levels.

Optionally, the method of the invention may comprise determining the level of one or more further markers and comparing the levels to control levels.

The control level in any of the methods discussed above may be determined from a sample from a healthy subject. According to this embodiment, a level of the at least one compound that is reduced compared to said control level is indicative of hepatic cancer.

Alternatively, the control level may be determined from a sample from a subject with hepatic cancer. According to this embodiment, a level of the at least one compound that is similar compared to said control level is indicative of hepatic cancer.

Although a sample from a control subject may be assayed in parallel to a sample from a test subject, it may be more convenient to use an absolute control level based on empirical data. An absolute control level provides a threshold such as a threshold level of at least one compound or a threshold profile level. The level of the at least one compound, or the profile level of a sample from a test subject may be compared to the threshold absolute control level wherein a level either higher or lower than the absolute control value is indicative of hepatic cancer.

The methods of the invention may be used to test samples from the same subject at two or more different points in time. Performing multiple test on the same subject over time allows the severity of disease to be measured, e.g. to observe whether the disease worsens. Alternatively, multiple testing may allow the efficacy of drugs to be monitored over time.

By glycine is meant a compound with the formula NH₂CH₂COOH or any naturally occurring variant thereof.

By trimethylamine-N-oxide is meant a compound with the formula (CH₃)₃NO or any naturally occurring variants thereof.

Herein, by hippurate it is meant a compound with the formula:

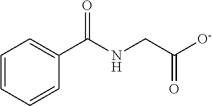 [SMILES: O=C([O-])CNC(=O)c1ccccc1]

or any naturally occurring variants thereof.

Herein, by citrate it is meant a compound with the formula:

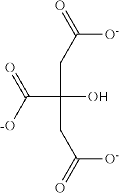

or any naturally occurring variants thereof.

Where any one of the above compounds is ionic, the counter ion may be any ion. Preferably the above compounds are in a neutral form.

### Hepatic Cancer

The hepatic cancer that may be detected by the methods of the invention may comprise any liver cancer. Hepatic cancer may comprise primary hepatic cancer including but not limited to hepatocellular carcinoma (HCC), fibrolamellar hepatocellular carcinoma, cholangiocarcinoma, angiosarcoma (or haemangiosarcoma) and hepatoblastoma. Alternatively hepatic cancer may comprise secondary hepatic cancer including cancer which has metastasised from the liver to other organs including but not limited to lung, kidney, breast, stomach and colon, skin (e.g. melanoma), prostate, pancreas and cervix.

### Subjects

A subject may be any animal e.g., a vertebrate or non-vertebrate animal. Vertebrate animals may be mammals. Vertebrate mammals may be human. Examples of mammals include but are not limited to mouse, rat, pig, dog, cat, rabbit, primate or the like. The subject may be a primate. Preferably the subject is human.

A test subject is a subject on which diagnosis is performed. The test subject may be a subject considered to be at risk of hepatic cancer. For example, the test subject may display symptoms of hepatic cancer such as jaundice, bloating from ascites, easy bruising from blood clotting abnormalities, loss of appetite, unintentional weight loss, abdominal pain, especially in the upper-right part, nausea, emesis, and fatigue. Alternatively the test subject may be considered to be at risk of hepatic cancer because they display genetic markers with a known link to hepatic cancer. Alternatively the test subject may be considered to be at risk of hepatic cancer because it tests positive for serum α-fetoprotein.

The methods of the invention may be used for analysing a sample from a subject which is a non-human animal, where the animal is used for screening of drugs for hepatic cancer. The effect of potential drugs on the level of the at least one compound may be indicative of whether the potential drug is efficacious.

The control subject is a subject against which the test subject is compared. The control subject may be a subject with hepatic cancer, in which case, a test subject displaying a similar profile to that of the control profile would be diagnosed as having hepatic cancer. Alternatively, the control subject may be a healthy subject, in which case, a test subject displaying a different profile to that of the control subject would be diagnosed as having hepatic cancer. Preferably, the control subject is a healthy subject.

A healthy subject may be any subject which does not have hepatic cancer. In a preferred embodiment the method of the invention is able to distinguish between a patient with hepatic cancer and a patient with cirrhosis. By determining the profile of a subject by considering the levels of at least one compound the method of the present invention allows for highly sensitive and specific testing which is able to differentiate between patients suffering from hepatic cancer and patients suffering from cirrhosis.

### Sample

The sample tested in the method of the invention may be any biological specimen obtained from a subject. A sample may be a tissue sample. The sample may be obtained with minimal invasiveness or non-invasively, e.g., the sample may be, or may be obtained from blood, plasma, serum, saliva, urine, stool, tears, any other bodily fluid, tissue samples (e.g., biopsy), and cellular extracts thereof (e.g., red blood cellular extract). One skilled in the art will appreciate that samples such as serum samples can be diluted prior to the analysis of levels of compounds.

Preferably the sample is a urine sample. The use of a urine sample in the method of the present invention is particularly advantageous since obtaining the sample from a subject is entirely non-invasive. This is useful where the subject may not wish to undergo invasive procedures in order to be tested for hepatic cancer, e.g. for ethical or religious reasons. Furthermore, the use of urine samples means that samples are obtained in a straightforward manner, and in some embodiments the method of the invention described above or the kit described below may be used to perform self testing.

### Further Compounds

The method of the invention may further comprise determining the level of at least one of the compounds selected from the group consisting of serum α-fetoprotein, creatinine, creatine, carnitine, acetone, lactate, glutamate, leucine, alanine, choline, phosphorylethanolamine, triglycerides, glucose, glycogen, acetate, N-acetylglycoproteins, pyruvate, glutamine, alpha-ketoglutarate, glycerol, tyrosine, 1-methylhistidine, phenylalanine, low-density lipoprotein, isoleucine, valine and acetoacetate, or any naturally occurring variants thereof in a sample and comparing the level of the at least one compound to at least one control level.

Measuring further compounds as part of the method of the invention allows the profile of the sample that may be produced to be more accurate, and therefore increases the sensitivity and specificity of the method. The more levels of compounds that are tested, the greater the capability of the method to distinguish subjects which display profiles similar to that of a subject with hepatic cancer but do not suffer from hepatic cancer e.g. cirrhosis sufferers.

In one embodiment, the method of the invention comprises determining the level of creatinine and comparing the level of creatinine with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of creatine and comparing the level of creatine with a control level, wherein the control level is determined from a sample a) from a healthy subject, wherein a level that is increased compared to said control level is indicative of hepatic cancer; and/or b) from a subject with hepatic cancer, wherein a level that is similar compared to said control level is indicative of hepatic cancer.

In one embodiment, the method of the invention comprises determining the level of carnitine and comparing the level of carnitine with a control level, wherein the control level is determined from a sample a) from a healthy subject, wherein a level that is increased compared to said control level is indicative of hepatic cancer; and/or b) from a subject with hepatic cancer, wherein a level that is similar compared to said control level is indicative of hepatic cancer.

In a preferred embodiment, the method of the invention comprises determining the level of glycine, trimethylamine-N-oxide, hippurate, citrate, creatinine, creatine and carnitine in a sample from a test subject and comparing the level of the compounds to a control level, wherein the control level is determined from a sample from a healthy subject, wherein a level that is reduced compared to said control level is indicative of hepatic cancer.

In another preferred embodiment, the method of the invention comprises determining the level of glycine, trimethylamine-N-oxide, hippurate, citrate, creatinine, creatine and carnitine in a sample from a test subject and comparing the level of the compounds to a control level, wherein the control level is determined from a sample from a hepatic cancer patient, wherein a level that is similar to said control level is indicative of hepatic cancer.

Herein, by creatinine it is meant a compound with the formula:

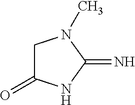

or any naturally occurring variants thereof.

Herein, by creatine, it is meant a compound with the formula:

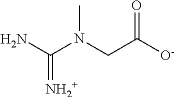

or any naturally occurring variants thereof.

Herein, by carnitine, it is meant a compound with the formula:

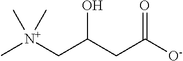

or any naturally occurring variants thereof. In one embodiment carnitine is the L-carnitine enantiomer.

Where any one of the above compounds is ionic, the counter ion may be any ion. Preferably the above compounds are in a neutral form.

### Determining the Level of a Compound

Herein, determining the level of a compound may be achieved using any quantitative or qualitative method known in the art whereby the level of at least one compound from a test sample can be compared to the level of at least one compound from a control sample.

Determination of the at least one level may be achieved by using a single method or a combination of methods.

Determination of the level of a compound my comprise determining a concentration of the compound, or alternatively may comprise determining the level of the compound on a relative scale.

Methods which may be used to determine the level of the at least one compound may include but are not limited to liquid chromatography, gas chromatography, high performance liquid chromatography (HPLC)¹⁴, capillary electrophoresis, as well as each of these techniques in combination with mass spectrometry, i.e. liquid chromatography-mass spectrometry¹⁵, gas chromatography-mass spectrometry¹⁶, high performance liquid chromatography-mass spectrometry, capillary electrophoresis-mass spectrometry¹⁷.

Other methods which may be used to determine the level of the at least one compound may include pyrolysis mass spectrometry, refractive index spectroscopy (RI), Ultra-Violet spectroscopy (UV), Near-InfraRed spectroscopy (Near-IR), microwave spectroscopy, Nuclear Magnetic Resonance spectroscopy (NMR)¹⁸, Raman spectroscopy, Light Scattering analysis (LS), thin layer chromatography (TLC), electrochemical analysis, fluorescence analysis, radiochemical analysis, nephelometry, turbidometry, electrical resistance analysis, fluid-solid interaction-based detection, spectrophotometry, colorimetry, optical reflection, heat-of combustion analysis, immunoassays, immunohistochemical assays, and other methods known in the art.

In one embodiment, determination of the level of the at least one compound will be achieved using a spectrophotometric assay. A spectrophotometric assay may be any assay wherein the quantity of a particular compound can be determined by measuring the capacity of a solution containing the substance to absorb light of particular wavelengths. A spectrophotmetric assay may comprise the direct detection of a compound present in a sample, where the compound provides a different absorbance at a known wavelength dependent on the level of compound present. Alternatively, the assay may involve the addition of reagents which undergo a change in absorbance in the presence of a particular compound. This change in absorbance may be measured in order to determine the level of the particular compound of interest.

### Colorimetric Assays

In one embodiment, determination of the level of the at least one compound may be achieved using a colorimetric assay. A colorimetric assay may be any assay in which the level of a compound may be determined by measuring or observing a colour change. A colorimetric assay may be a spectrophotometric assay wherein the wavelength at which the absorbance of the substance is measured is within the visible region of the electromagnetic spectrum. Colorimetric assays may comprise the comparison of the colour of a sample with a colour chart. Colorimetric assays may comprise the addition of reagents that undergo a measurable colour change in the presence of a particular compound.

The determination of the level of each of the at least one compounds may be determined using a separate colorimetric assay. Any colorimetric assay may be used for the determination of the level of a compound wherein the assay causes a colour change that is dependent only on the level of the compound in the sample and is not to a significant degree dependent upon any other variables.

Any assay known in the art may be used to detect the level of at least one compound in a sample. Examples of assays that may be used are:

Citrate assays such as those available from AbCam (product code—ab83396) which involve converting citrate to pyruvate via oxaloacetate. Pyruvate subsequently converts a colourless probe into an easily detectable intensely coloured (Lambda max=570 nm) and fluorescent (Ex/Em, 535/587 nm) product.

Glycine assays which involve the degradation of glycine to formaldehyde by chloramine T. The formaldehyde may then be converted to 3,5-diacetyl-1,4-dihydrolutidine by the Hantzsch reaction in which acetyl acetone and ammonia are the reactants. This reaction product in low ranges (level of glycine from 0.1 to 3 g), can be measured fluorometrically, whereas in higher ranges colorimetric analysis may be used¹⁹.

TMAO assays involving reduction to TMA by an equimolar mixture of ferrous sulfate (FeSO₄) and disodium ethylenediaminetetraacetic acid (EDTA) (0.1 M) in acetate buffer (0.8 M, pH 4.5) followed by the use of picrate as a chromagen^{20,21}.

### Human or Machine Readable Strips

The level of the at least one compound may be determined through use of a human or machine readable strip, in which the level of said at least one compound, may be determined by measuring a change in said human or machine readable strip. A change in the human or machine readable strip may be a change in the human or machine readable strip which occurs via a chemical reaction between a reagent present in or on said human or machine readable strip and said at least one compound. For example, the human or machine readable strip may comprise reagents for performing at least one colorimetric assay to determine the level of at least one compound.

The human or machine readable strip may comprise multiple regions, wherein a separate chemical reaction is conducted in each region. The chemical reaction in each region may be used to detect one of the at least one compounds. On contact with the sample, the reagents present in each region are able to undergo chemical reactions with compounds present in the sample. The levels of each of the at least one compounds may then be determined according to the degree of change, e.g. a colour change that has taken place in each region of the human or machine readable strip.

In one embodiment, where the change in the human or machine readable strip is a colour change, the human or machine readable strip may be read by a human comparing the human or machine readable strip with a chart which shows varying degrees of colour, and which attributes the varying degrees of colour with particular levels of compound.

In another embodiment, the human or machine readable strip may be read by a machine which calculates the degree of change, e.g. a colour change, that has occurred in the human or machine readable strip in each region either by measuring the absorbance of the solution at a particular wavelength or by any other means.

The assays provided in each region of the human or machine readable strip may be any assay which involves a change occurring in a region of the human or machine readable strip wherein the change is dependent only on the level of the at least one compounds.

### Binding Assays

The level of the at least one compound may be determined by a method involving a binding assay. A binding assay may be any assay where one of the at least one compounds is specifically bound by one or more other molecules wherein the one or more other molecules may subsequently be detected. In one embodiment, the one or more other molecules may be one or more proteins. Proteins which specifically bind one of the at least one compounds may subsequently be detected using an antibody specific to the one or more proteins.

In one embodiment, the one or more other molecules may be antibodies which specifically bind to one of the at least one compounds.

An antibody may be a monoclonal or a polyclonal antibody or a fragment thereof.

The level of the at least one compound may be determined using an immunoassay or an immunohistochemical assay. Examples of immunohistochemical assays suitable for use in the method of the present invention include, but are not limited to, immunofluorescence assays such as direct fluorescent antibody assays, indirect fluorescent antibody (IFA) assays, anticomplement immunofluorescence assays, and avidin-biotin immunofluorescence assays. Other types of immunohistochemical assays include immunoperoxidase assays.

The level of the at least one compound may be determined using an antibody which specifically binds to said compound, and said antibody may be detected through colorimetric or radiometric means otherwise known in the art. In one embodiment, the level of the at least one compound may be determined using a sandwich assay, whereby one of the at least one compounds is specifically bound by one protein, and specifically detected by a second protein.

The level of a compound may be determined by providing a binding protein which is known to specifically bind to said compound. Said binding protein may subsequently be detected by an antibody specific to said binding protein.

### Profiling the Sample

A profile of the sample may be determined by analysing the level of the at least one compound. Profiling the sample allows differences between groups of subjects to be characterised by a combination of metabolite ratios (a “metabolic profile”) rather than a single metabolite.

A profile of the sample may be used to determine, on the basis of the levels of the at least one compound, whether the subject has hepatic cancer. A profile of the sample allows for an overall comparison of the levels of compounds in a sample from a test subject and a sample from a control subject.

Profiling may involve normalisation which may include consideration of the level of the at least one compound relative to external compounds, e.g. urinary creatinine. This may for example normalise the levels depending on the dilution of the sample by comparing levels of other compounds which are not altered in subjects with hepatic cancer compared to healthy subjects. Alternatively, profiling may involve calculating the ratios of the at least one compounds. Profiling may give more weight to certain compounds of the at least one compounds compared to others.

### Kits

The invention provides a kit for use in diagnosing hepatic cancer, the kit comprising at least one reagent for determining the level of a compound selected from the group consisting of glycine, trimethylamine-N-oxide, hippurate and citrate.

The kit of the invention may comprise at least two reagents for determining the level of a compound selected from the group consisting of glycine, trimethylamine-N-oxide, hippurate and citrate.

The kit of the invention may comprise at least three reagents for determining the level of a compound selected from the group consisting of glycine, trimethylamine-N-oxide, hippurate and citrate.

The kit of the invention may comprise reagents for determining the level of glycine, trimethylamine-N-oxide, hippurate and citrate.

The reagents used in the kits of the invention for determining the level of the at least one compound may cause a colour change in the assay dependent of the level of the at least one compound.

The reagents used in the kits of the invention for determining the level of the at least one compound may comprise at least one antibody which binds specifically to the at least one compound.

The kits of the invention may further comprise instructions for use.

The kits of the invention may comprise reagents for determining the level of the at least one compound placed on a test strip. Different regions of the test strip may comprise reagents for determining the level of multiple compounds and therefore different assays may be performed in different regions of the test strip.

In one embodiment a kit of the invention may comprise a test strip comprising reagents for determining the level of at least one compound, and a comparison chart. A comparison chart may shows varying degrees of colour against which the colour present on the test strip can be measured. The comparison chart may attribute the varying degrees of colour which may be present on the test strip with particular levels of compound.

### BRIEF DESCRIPTION OF THE FIGURES

FIG. 1: Median 1H NMR spectra from A. Patients with HCC; B. Patients with cirrhosis and C. Healthy controls

FIG. 2A: Principal components analysis (PCA) scores plots of HCC subjects versus healthy controls

FIG. 2B: Principal components analysis (PCA) scores plots of HCC versus cirrhosis subjects

FIG. 2C: Orthogonal signal correction—partial least squared discriminant analysis (OSC-PLS-DA) of healthy subjects vs. HCC subjects

FIG. 2D: Orthogonal signal correction—partial least squared discriminant analysis (OSC-PLS-DA) of HCC subjects vs. cirrhosis subjects

FIG. 3: Median integral value of A. Creatinine; B. Citrate; C. Carnitine; D. Creatine; E. Hippurate; F. Glycine; G. Trimethylamine-N-Oxide in samples from healthy subjects, cirrhosis subjects and HCC subjects

### EXAMPLES

### Example 1

### Patient Selection

Egyptian urine and serum samples were collected from patients attending the National liver Institute, Menoufiya University, Shbeen El Kom, Egypt. Ethical approval was granted by the research ethics committees at the National Liver Institute, Menoufiya University and the Hammersmith Hospital Campus, Imperial College London.

A total of 58 patients were recruited for study: 18 patients with HCC (diagnosed by two imaging techniques showing early arterial enhancement and rapid washout in portal phase or one imaging with serum AFP >400 ngmL⁻¹); 20 patients with clinically or histologically-confirmed cirrhotic liver disease; and 20 healthy Egyptian control subjects. Eleven samples from all three cohorts were identified as “outliers” according to principal component analysis (PCA), a multivariate analytical technique. This was indicative of aberrant spectral findings which unfairly influenced the multivariate data and these samples were excluded from further analysis. This left 16 samples in the HCC group, 14 in the cirrhosis group and 17 in the healthy control group. The median age of the HCC group was not significantly older than the cirrhosis group (p=0.37), but was significantly older than the healthy controls (p=0.01). There were significantly more males in the HCC group (15/16) than the healthy control group (9/17) (p=0.02), but not compared to the cirrhosis group (11/14) (p=0.32). Staging of HCC stage was according to the Okuda staging system, based on tumour volume and hepatic decompensation, stage I representing early disease and stage III, advanced disease (28). One of the HCC subjects had stage I disease, 10 stage II and 5 stage III. The majority of patients with HCC and 50% of patients with cirrhosis were HCV antibody positive: 11/16 (69%) and 7/14 (50%), respectively. The remaining patients with HCC or cirrhosis had diverse aetiologies. All healthy controls were of Egyptian origin and had no history of liver disease.

### Example 2

### Urine Sample Collection

Random 5 mL urine samples were collected into plain glass tubes and stored at −80° C., 2 hrs to 4 hrs after collection in Egypt, until air transportation on dry ice to Imperial by A.I.G, M.M.E.C and S.D.T-R. Samples were thawed in London and prepared according to standard methodology²²: 400 μL of urine were mixed with 200 μL of buffer solution (0.2 M Na₂HPO₄/0.2 M NaH₂PO₄, pH 7.4), and 60 μL of 3-trimethylsilyl-(2,2,3,3-²H₄)-1-propionate (TSP)/D₂O solution (final concentration of TSP=1 mM) were added. The TSP served as an internal chemical shift reference (δ 0.00 ppm) and the D₂O provided a field lock. The buffered urine sample was left to stand for 10 min and then centrifuged at 13,000 g for 10 min. 550 μL of supernatant were transferred into a 5 mm diameter glass NMR tube (Wilmad LabGlass™, New Jersey, USA) for proton nuclear magnetic resonance (¹H NMR) spectroscopy. Samples remained in a sample queue on the NMR auto-analyser for up to 4 hrs until data acquisition

### Example 3

### Serum Laboratory Tests

### Method

Serum AFP, creatinine, alanine transaminase (ALT), aspartate transaminase (AST), bilirubin and albumin were measured at the time of urine sample collection in Egypt using a Cobas Integra 400-Autoanalyzer, (Roche, Rotkreuz, Switzerland).

### Results

A summary of median (range) values for serum AFP, creatinine, ALT, AST, bilirubin and albumin of study subjects are displayed in Table 1. Serum AFP levels were only measured in the cirrhosis and HCC groups. Although median serum AFP was significantly higher in HCC patients (745 IU mL⁻¹ versus 77 IU mL⁻¹, p<0.001), the lowest serum AFP in the cirrhosis group was 20 IU mL⁻¹ and using a cut-off of 20 IU mL⁻¹ all cirrhosis patients would have been falsely classified as HCC based on serum AFP levels. Median serum creatinine was significantly higher in the HCC group compared to controls (106 mmol L⁻¹ versus 62 mmol L⁻¹, p<0.001) but this was not significantly different to cirrhosis subjects (106 mmol L⁻¹ versus 150 mmol L⁻¹, p=0.16). Serum ALT, AST and bilirubin levels were all significantly higher in the HCC group compared to healthy controls but not compared to cirrhosis subjects. Serum albumin was significantly lower in the HCC group compared to healthy controls but not compared to cirrhosis subjects.

TABLE 1

| | | | Healthy | |
|---|---|---|---|---|
| | | Cirrhosis | Controls | p-values (Mann- |
| | HCC (range) | (range) | (range) | Whitney) |
| Serum Samples (n) | 16 | 14 | 17 | — |
| AFP (IU mL⁻¹) | 745 (174-5000) | 77 (20-174) | — | ^{b}<0.001 |
| Creatinine (mmol L⁻¹) | 106 (44-318) | 150* (97-229) | 62 (44-80) | ^{a}<0.001 and ^{b}0.16 |
| ALT (IU L⁻¹) | 56 (11-164) | 30 (16-119) | 12 (9-18) | ^{a}<0.001 and ^{b}0.36 |
| AST (IU L⁻¹) | 79 (22-224) | 51 (31-190) | 15 (11-20) | ^{a}<0.001 and ^{b}0.44 |
| Bilirubin (μmol L⁻¹) | 63 (17-547) | 48 (14-156) | 12 (0.7-19) | ^{a}<0.001 and ^{b}0.19 |
| Albumin (g L⁻¹) | 30 (19-34) | 29 (23-35) | 40 (38-44) | ^{a}<0.001 and ^{b}0.79 |

*Two samples did not have serum creatinine levels measure
Key:
^{a}HCC versus healthy controls;
^{b}HCC versus cirrhosis.

### Example 4

### ¹H NMR Spectroscopy Spectral Acquisition and Processing

Samples were run in a random, non-grouped order. ¹H MR spectra were acquired using a pulse-collect sequence with water presaturation (JEOL 500 MHz Eclipse+NMR spectrometer). The 16 data collects were summated. A 90° pulse angle was used with a total repetition time of 18.7 s, so that fully-relaxed data were acquired. The acquisition time was 8.7 s. Data were collected into 64K points. MR spectra were processed using the KnowItAll™ Informatics System v7.8 (Bio-Rad, Philadelphia, USA). Free induction decays were zero-filled by a factor of two and multiplied by an exponential window function with a 0.3 Hz line-broadening factor, prior to Fourier transformation. All MR spectra were phased and a baseline correction applied. All spectra were referenced to TSP (δ 0.00 ppm) and the methyl-creatinine peak was aligned to δ 3.05 ppm. MR spectral resonances were assigned according to literature^{23,24,25}. MR spectral analysis included the range δ 0.20-10.00 ppm, excluding the region δ 4.50-6.40 ppm, to remove the residual water signal and variation in urea signal due to exchange of protons within the solvent.

### Example 5

### Multivariate Statistical Analysis

### Method

Differences between patient groups were characterised using a combination of metabolite ratios (a “metabolic profile”) rather than a single metabolite. Multivariate statistical analysis in the form of principal components analysis (PCA) and partial least squared discriminant analysis (PLS-DA) were used for initial analysis²⁶. PCA is an unsupervised analytical tool that provides an overview of complex data through an examination of the covariance structure, highlighting sample outliers and clustering. PLS-DA is a supervised analytical method that relates metabolite data to class membership, elucidating separation between the groups. The complex spectra were divided into smaller regions or “buckets” of 0.02+/−0.01 ppm, representing specific metabolites peaks, using the “intelligent bucketing” algorithm in the software application KnowltAll Informatics System v7.8 (Bio-Rad, Philadelphia, USA). These regions were then integrated, normalised to the sum of the total spectral integral and the data mean-centred prior to multivariate analysis. Pareto-scaled data were also used, though the results were similar to mean-centred data and had the tendency to model spectral noise, therefore, only mean-centred data were used for all analyses. PCA was performed using the same software to highlight clustering and outliers. Data were then analysed by PLS-DA using Pirouette v4.0 (Infometrix, Washington, USA). The data filtering technique of orthogonal signal correction (OSC) was used to remove variation in the spectra not directly related to the physiological condition being studied and to minimise the possible influence of inter-individual variation^{27,28}. For each analysis, one OSC component was removed for model generation. The discriminatory power of each model was validated using two techniques. First, leave-one-out cross validation, whereby each sample in turn was excluded from the analysis, a model created from the remaining samples and the class membership of the excluded sample predicted²⁹. Second, full external validation in which models were created from training sets (70% of the samples, selected at random) and their predictive power tested using independent “test” sets (the held-back 30% of the samples). Full external validation was repeated three times, excluding a different set of 30% of samples and the mean taken. For both techniques, the misclassification matrix of the model described the number of correctly predicted samples, and from this the sensitivity and specificity of the model could be calculated.

### Results

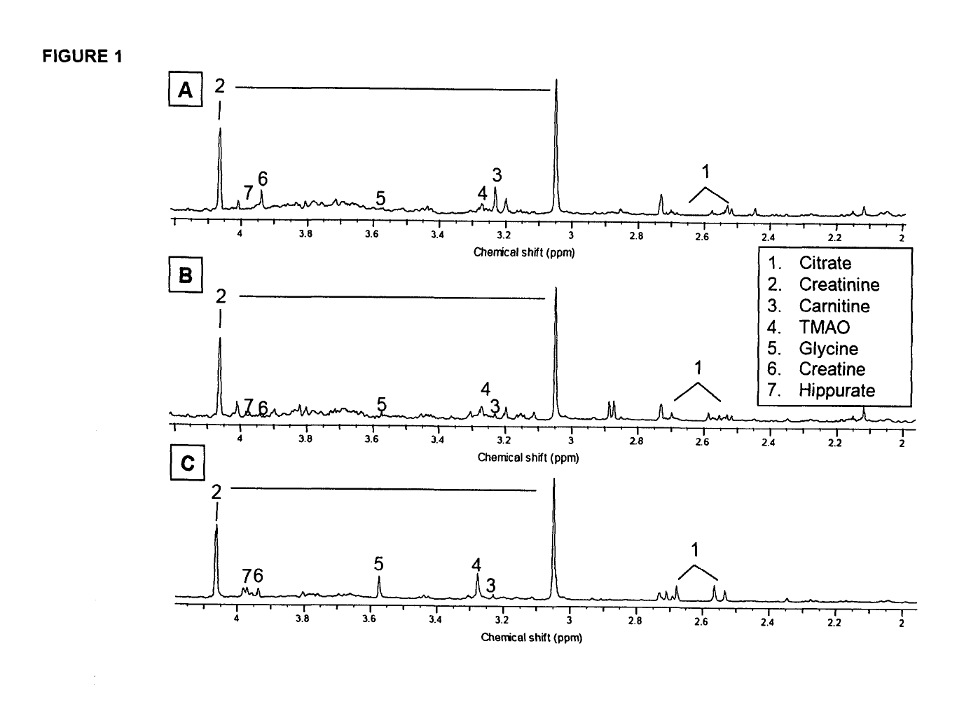

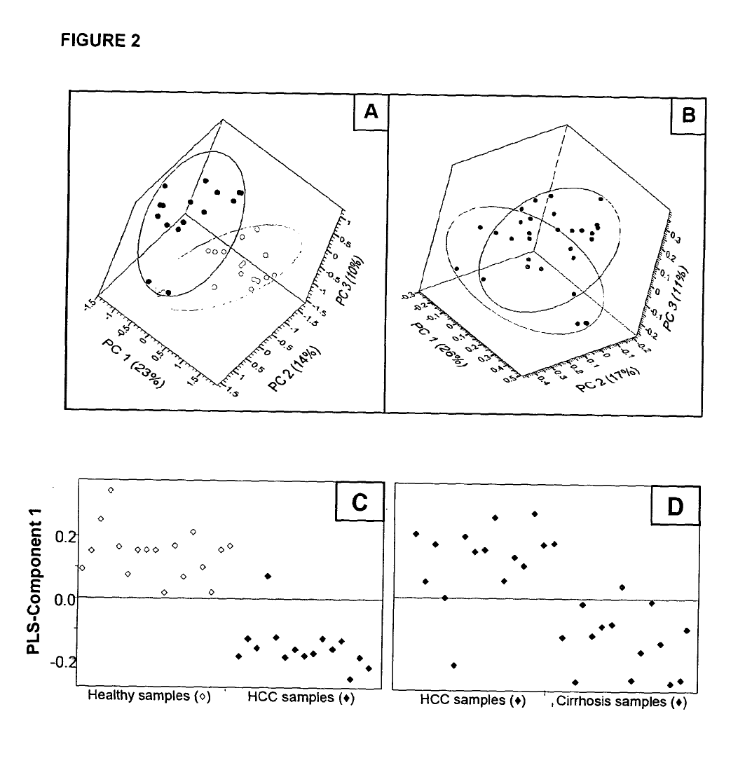

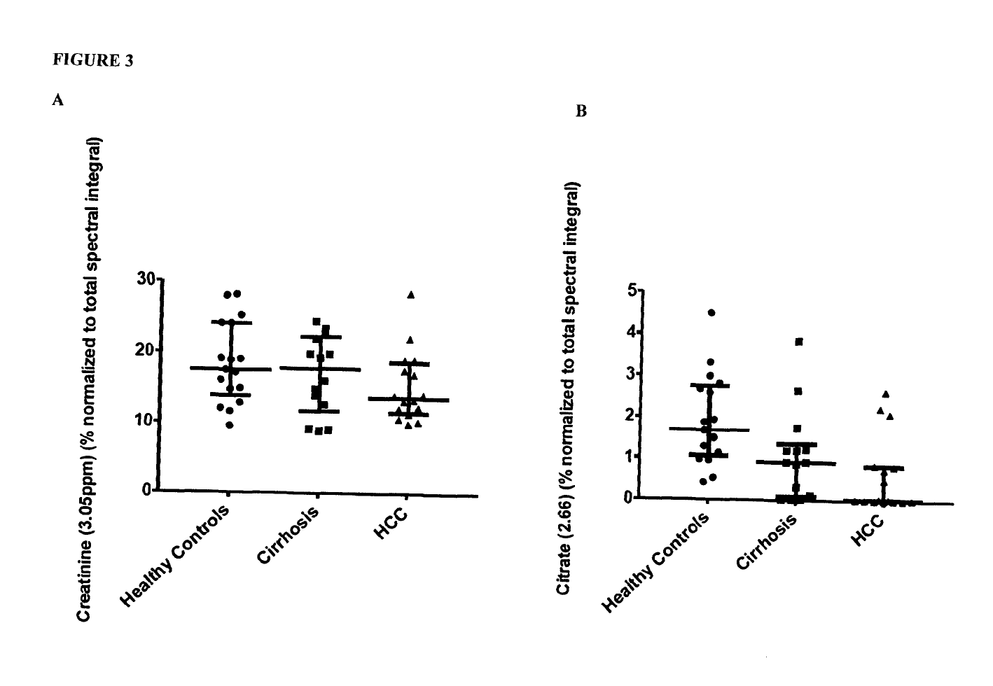

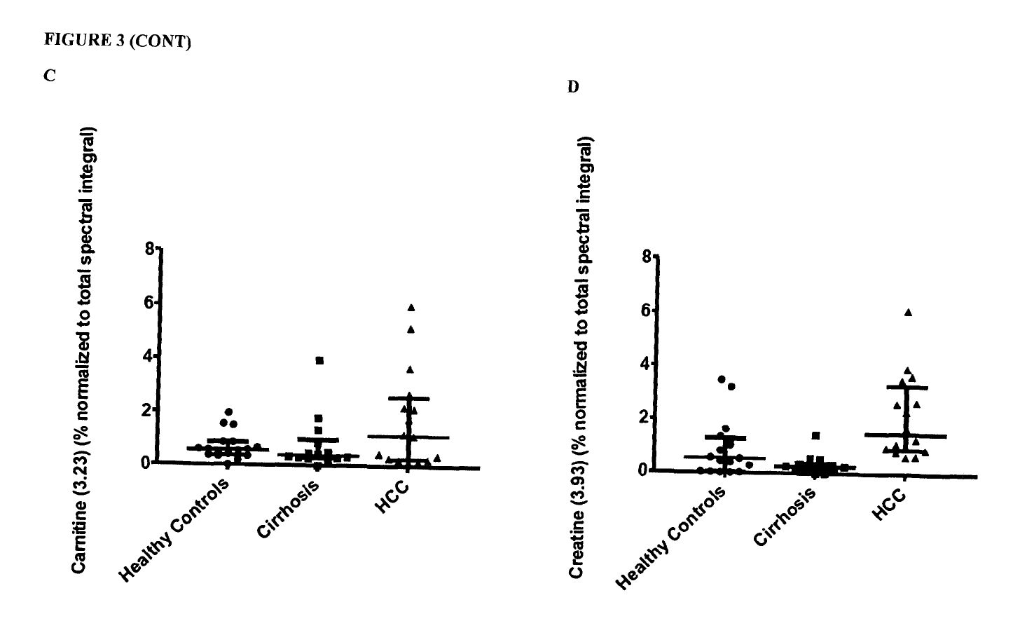

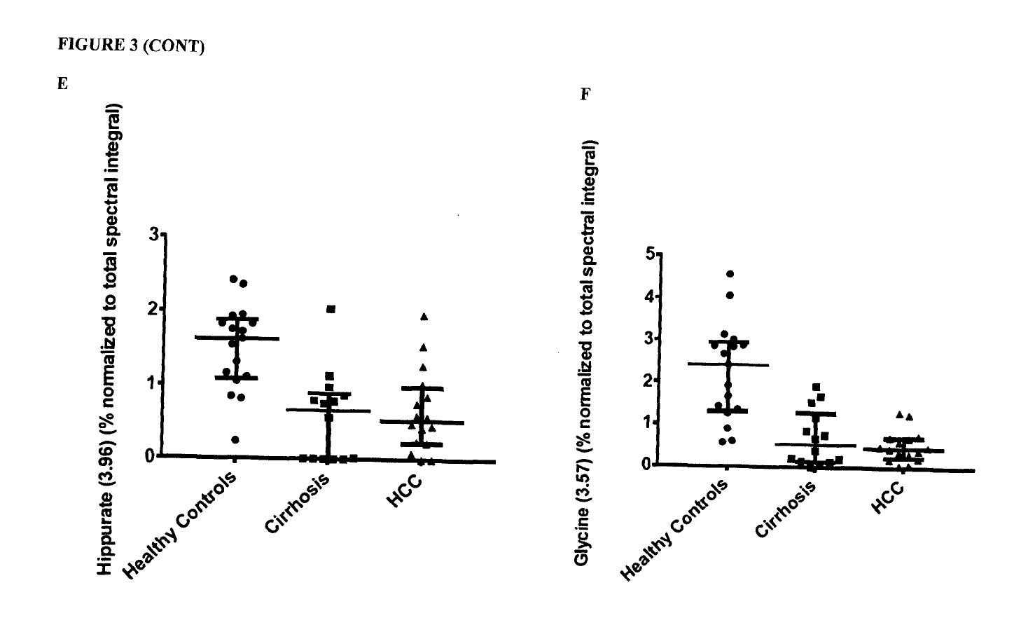

Representative urinary spectra from the three subject cohorts are displayed in FIG. 1. Eleven samples were identified as “outliers” by PCA and excluded from further analysis. These included two sample spectra from the HCC group: one of which displayed dominant glucose metabolites, indicating glycosuria which may have resulted from undiagnosed diabetes mellitus and one sample which could not be phased adequately; six spectra from the cirrhosis group: three of which displayed marked glucose metabolites, one which could not be phased adequately, one with dominant lactate peaks and one sample which displayed pronounced unidentified peaks at δ1.45 ppm and δ1.60 ppm which strongly affected multivariate analyses; and three spectra from the healthy control group all of which displayed dominant glucose metabolites indicating glycosuria. PCA scores plots of the resulting groups showed clear clustering of HCC versus healthy controls samples (FIG. 2A). OSC-PLS-DA distinguished HCC and healthy control cohorts with 100% sensitivity and 94% specificity using a leave one out algorithm (R²=0.8, Q²=0.74) (FIG. 2C). Using a full external validation paradigm, discriminatory sensitivity was 100% and specificity 93%. Resonances contributing most to the PLS-DA models were glycine (δ 3.57 ppm), trimethylamine-N-oxide (TMAO) (δ 3.27 ppm), hippurate (δ3.96 ppm), citrate (δ 2.66 ppm), creatinine (δ 3.05 ppm), creatine (δ 3.93 ppm) and carnitine (δ 3.23 ppm).

PCA scores plots of HCC versus cirrhosis samples displayed poorer group clustering (FIG. 2B). OSC-PLS-DA distinguished HCC and cirrhosis cohorts with 81% sensitivity and 71% specificity using a leave one out algorithm (R²=0.54, Q²=0.25) (FIG. 2D). Using a full external validation paradigm, discriminatory sensitivity was 75% and specificity 67%. Resonances contributing most strongly to PLS-DA discrimination were TMAO (δ 3.27 ppm), creatine (δ 3.93 ppm) and carnitine (δ 3.23 ppm).

Owing to the male predominance of samples in the liver disease group, male only analyses were undertaken to confirm that findings were due to the effects of HCC rather than gender. Male HCC urine could be discriminated with 100% sensitivity and 93% specificity from healthy control urine and with 80% sensitivity and 55% specificity from urine of patients with cirrhosis. Discriminatory metabolites were similar to combined gender comparisons: glycine, TMAO, hippurate, citrate, creatinine, creatine, and carnitine.

### Example 6

### Univariate Statistical Analysis

### Method

The most important discriminatory metabolite resonances, as determined by PLS-DA loadings plots were integrated and normalised to the sum of the total spectral integral. Values were expressed as percentage index, relative to the total spectral integral. Using GraphPad Prism v5.01 (California, USA), differences between the HCC, cirrhosis and healthy control groups were analysed using the Mann-Whitney test, assuming non-normal data distribution, p-values <0.05 were considered significant.

### Results

### Metabolites Reduced in HCC Subject Urine

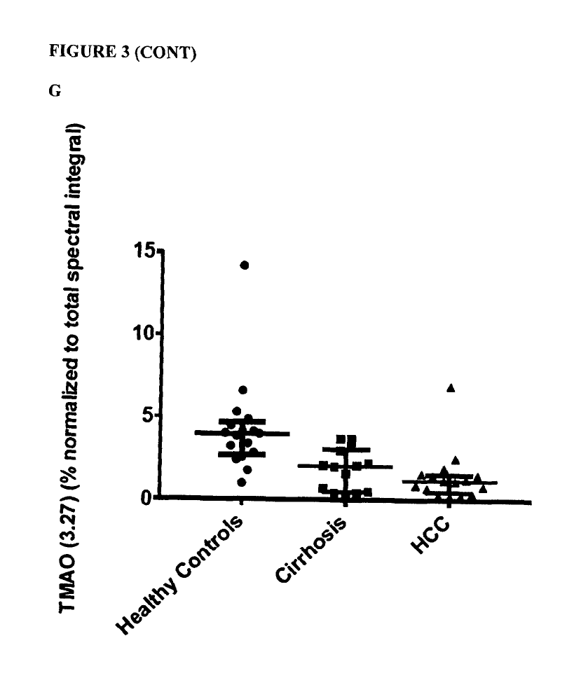

Urinary glycine levels (expressed as % normalized to total spectral integral) were significantly reduced in HCC subjects urine compared to healthy controls: median [interquartile range]0.46 [0.24-0.71] and 2.41 [1.30-2.95](p<0.001), but were not significantly reduced compared to cirrhosis subjects: 0.53 [0.13-1.27](p=0.88) (FIG. 3F). Urinary TMAO levels were significantly reduced in HCC subject urine compared to healthy controls: 1.17 [0.47-1.56] and 3.98 [2.69-4.67](p<0.001), but non-significantly reduced compared to cirrhosis subjects: 2.03 [0.47-3.07](p=0.18) (FIG. 3G). Urinary hippurate levels were significantly reduced in HCC subjects compared to healthy subjects: 0.53 [0.23-0.98] and 1.62 [1.08-1.88](p<0.001), but not significantly different compared to cirrhosis subjects: 0.66 [0.0-0.89](p=0.65) (FIG. 3E). Urinary citrate was significantly reduced compared to healthy subjects: 0.04 [0.0-0.84] and 1.68 [1.06-2.74](p<0.001), with a reduced trend towards significance compared to cirrhosis subjects: 0.91 [0.09-1.36](p=0.12) (FIG. 3B). Urinary creatinine levels were reduced in HCC urine compared to healthy control and cirrhosis subjects, but not to a level of significance: 13.7 [11.59-18.69], 17.49 [13.82-24.11] and 17.71 [11.78-22.28](p=0.12 and 0.33, respectively) (FIG. 3A).

### Metabolites Elevated in HCC Subject Urine

Urinary creatine levels were significantly elevated in HCC subject urine, compared to healthy controls and cirrhosis subjects: 1.5 [0.92-3.32]; 0.54 [0.04-1.28] and 0.26 [0.12-0.39](p=0.003 and p<0.001, respectively) (FIG. 3D). Urinary carnitine levels were non-significantly elevated in HCC subject urine, compared to healthy controls and cirrhosis subjects: 1.16 [0.25-2.59]; 0.58 [0.36-0.86] and 0.39 [0.29-0.97](p=0.29 and p=0.30) (FIG. 3C).

### REFERENCES

¹ El-Serag, H. B. Hepatol. Res. (Suppl. 2) S88-S94
² Taylor-Robinson, S. D. et al., Lancet 1997, 350 (9085), 1142-1143
³ Khan, S. A, et al., J. Hepatol. 2002, 37 (6), 806-813
⁴ Shariff M I et al., Expert Rev Gastroenterol Hepatol. 2009, 3 (4), 353-367
⁵ www.hepatocellular.org
⁶ Chien-Jen Chen et al., JAMA 2006, 295 (1): pp. 65-73.
⁷ Furui J, Furukawa M, Kanematsu T. The low positive rate of serum alpha-fetoprotein levels in hepatitis C virus antibody-positive patients with hepatocellular carcinoma. Hepatogastroenterology 1995 September; 42(5):445-449.
⁸ Nguyen M H, Keeffe E B. Screening for hepatocellular carcinoma. J Clin Gastroenterol 2002 November; 35(5 Suppl 2):S86-S91.
⁹ Peng Y C, Chan C S, Chen G H. The effectiveness of serum alpha-fetoprotein level in anti-HCV positive patients for screening hepatocellular carcinoma. Hepatogastroenterology 1999 November; 46(30):3208-3211.
¹⁰ Stefaniuk P, Cianciara J, Wiercinska-Drapalo A. Present and future possibilities for early diagnosis of hepatocellular carcinoma. World J Gastroenterol 2010 Jan. 28; 16(4):418-424.
¹¹ Raedle, J. et al., C. Dig. Dis. Sci 1995, 40 (12), 2587-2594.
¹² Gomaa et al., World J. Gastroenterol., in press.
¹³ El-Serag H B, Marrero J A, Rudolph L, Reddy K R (May 2008). “Diagnosis and treatment of hepatocellular carcinoma”. Gastroenterology 134 (6): 1752-63.
¹⁴ Yang J., et al. J. Chromoatogra B, 2004, 813, 1-2, 59-65
¹⁵ Lu, X. et al., J. Chromatogra. B, 2008, 866, 64
¹⁶ Pasikanti, K. K. et al., J. Chromatagr. B, 2008, 871, 202
¹⁷ Benavente F. et al., Electrophoresis, 2006, 27, 4570
¹⁸ Hollywood, K. et al., Proteomics 6, 2006, 4716
¹⁹ Sardesai M., et al., Clinica Chimica Acta, 1970, 29, 1, 67-71
²⁰ Barnes and Blackstock, 1974
²¹ Wekell J. C. et al., Journal of food science, 1991, 56(1), 132-135
²² Beckonert O, Keun H C, Ebbels T M, Bundy J, Holmes E, Lindon J C, et al. Metabolic profiling, metabolomic and metabonomic procedures for NMR spectroscopy of urine, plasma, serum and tissue extracts. Nat Protoc 2007; 2(11):2692-2703.
²³ Holmes E, Foxall P J, Spraul M, Farrant R D, Nicholson J K, Lindon J C. 750 MHz 1H NMR spectroscopy characterisation of the complex metabolic pattern of urine from patients with inborn errors of metabolism: 2-hydroxyglutaric aciduria and maple syrup urine disease. J Pharm Biomed Anal 1997 July; 15(1):1647-1659.
²⁴ Lindon J C, Holmes E, Nicholson J K. NMR spectroscopy of biofluids. Annu Rep NMR Spectrosc 1999; 6(2):1-88.
²⁵ Wishart D S, Tzur D, Knox C, Eisner R, Guo A C, Young N, et al. HMDB: the Human Metabolome Database. Nucleic Acids Res 2007 January; 35 (Database issue):D521-D526
²⁶ Trygg J, Holmes E, Lundstedt T. Chemometrics in metabonomics. J Proteome Res 2007 February; 6(2):469-479.
²⁷ Psihogios N G, Gazi I F, Elisaf M S, Seferiadis K I, Bairaktari E T. Gender-related and age-related urinalysis of healthy subjects by NMR-based metabonomics. NMR Biomed 2008 March; 21(3):195-207.
²⁸ Westerhuis J, Hoefsloot H, Smit S, Vis D, Smilde A, van Velzen J, et al. Assessment of PLSDA cross validation [Abstract]. Metabolomics 2008 March 24; 4(1):81-89.
²⁹ Mahadevan S, Shah S L, Marrie T J, Slupsky C M. Analysis of metabolomic data using support vector machines. Anal Chem 2008 October 1; 80(19):7562-7570

1. A method for analysing a sample from a test subject comprising:
i) determining the level of at least one compound selected from the group consisting of glycine, trimethylamine-N-oxide, hippurate and citrate in the sample from the test subject; and
ii) comparing the level of the at least one compound determined in step i) to at least one control level, wherein the levels of the at least one compound are indicative of whether the subject has hepatic cancer.

2. The method of claim 1, wherein the control level is determined from a sample from a healthy subject, wherein a level that is reduced compared to said control level is indicative of hepatic cancer.

3. The method of claim 1, wherein the control level is determined from a sample from a hepatic cancer patient, wherein a level that is similar to said control level is indicative of hepatic cancer.

4. The method of claim 1, comprising determining the level of at least two compounds selected from the group consisting of glycine, trimethylamine-N-oxide, hippurate and citrate in a sample.

5. The method of claim 1, comprising determining the level of at least three compounds selected from the group consisting of glycine, trimethylamine-N-oxide, hippurate and citrate in a sample.

6. The method of claim 1, comprising determining the level of glycine, trimethylamine-N-oxide, hippurate and citrate in a sample.

7. The method of claim 1, comprising determining a profile for the sample from the test subject using the level of the at least one compound; and comparing the profile of the sample from the test subject with a control profile determined using at least one control level determined from a sample from a healthy subject.

8. The method of claim 1, further comprising
a) determining the level of at least one further compound selected from the group consisting of creatinine, creatine, carnitine and acetone; and
b) comparing the level of the at least one compound determined in step a) to a control level determined from a sample from a healthy subject, wherein
i) a reduction in the level of creatinine and/or acetone; and/or
ii) an increase in the level of creatine and/or carnitine
is indicative of hepatic cancer.

9. The method of claim 1, wherein the sample is obtained from a mammal.

10. The method of claim 9, wherein the mammal is a human.

11. The method of claim 1, wherein the sample is selected blood, blood plasma, blood serum, cerebrospinal fluid, bile acid, saliva, synovial fluid, pleural fluid, pericardial fluid, peritoneal fluid, feces, nasal fluid, ocular fluid, intracellular fluid, intercellular fluid, lymph fluid, and urine.

12. The method of claim 11 wherein the sample is urine.

13. The method of claim 1, wherein the hepatic cancer is hepatocellular carcinoma.

14. The method of claim 1, wherein determining the level of a compound comprises contacting the sample with an antibody which binds specifically to said compound.

15. The method of claim 1, wherein determining the level of a compound comprises performing a colorimetric or spectrometric assay on the sample.

16. The method of claim 1, wherein the method is able to distinguish between a patient with hepatic cancer and a patient with cirrhosis.

17. A kit for use in diagnosing hepatic cancer, the kit comprising at least one reagent for determining the level of a compound selected from the group consisting of glycine, trimethylamine-N-oxide, hippurate and citrate.

18. The kit of claim 17, comprising at least two reagents for determining the level of a compound selected from the group consisting of glycine, trimethylamine-N-oxide, hippurate and citrate.

19. The kit of claim 17, comprising at least three reagents for determining the level of a compound selected from the group consisting of glycine, trimethylamine-N-oxide, hippurate and citrate.

20. The kit of claim 17, comprising reagents for determining the level glycine, trimethylamine-N-oxide, hippurate and citrate.

21. The kit of claim 17, wherein the reagents for determining the level of the at least one compound cause a colour change in the assay dependent of the level of the at least one compound.

22. The kit of claim 17, wherein the reagents for determining the level of the at least one compound comprise at least one antibody which binds specifically to the at least one compound.
